(19) United States
(12) Patent Application Publication
(10) Pub. No.: US 2021/0308394 A1
(43) Pub. Date: Oct. 7, 2021

(54) A DELIVERY SYSTEM FOR AROMATIC OILS

(71) Applicant: BTI Therapeutics Inc, Boston, MA (US)

(72) Inventor: Shlomi Hollander, Zichron Yaakov (IL)

(21) Appl. No.: 17/270,025

(22) PCT Filed: Aug. 29, 2019
(86) PCT No.: PCT/IB2019/057268

§ 371 Date: Feb. 21, 2021

Related U.S. Application Data
(60) Provisional application No. 62/724,641, filed on Aug. 30, 2018.

(51) Int. Cl. A61M 15/08; A61M 15/00; A61M 21/00

(52) U.S. Cl. CPC A61M 15/085; A61M 2021/0016; A61M 21/00; A61M 15/0061

(57) ABSTRACT

The present invention provides a system for delivering oils to a person's nasal passage without the oils coming into contact with the sensitive mucous membranes of the nostrils of the user. The delivery system includes a nasal clip, externally attached to a nasal septum of a user, and a pair of scented inserts which are configured to fit and be secured by a pair of generally cylindrical shaped receptacles formed at the end of the nasal clip.

### FIELD OF THE INVENTION

The present invention generally relates to a system and apparatus for delivering oils, such as aromatic oils to a person's nasal passages.

### BACKGROUND OF THE INVENTION

The use of aromatic oils to improve the well-being of people suffering from nausea, allergies and discomfort are well known in the art. One popular method includes the use of nasal clips for disseminating aromatic substances.

U.S. Pat. No. 7,108,198 to Altadonna describes an intra nasal dispenser clip for aromatherapy and for dispensing fragrances.

US Published application No: 2016/0367773 to Davi describes a nasal clip characterized in being made from a heat sensitive plastic having a volatile aromatic material diffused therein.

U.S. Pat. No. 7,837,649 to Aboff describes the use of a nasal clip containing a holster cavity for holding a scented bar impregnated with aromatic substances.

The purpose of the holster cavity of U.S. Pat. No. 7,837,649 is to keep the scent bar away from the skin, since the oils can irritate the skin if in direct contact. The base is fashioned in a way with various openings in order to emit as much aroma as possible. However, the effectiveness of the product is limited, since the diffusion of the aromatic oils is restricted by the holster itself.

The prior art nasal clips are inefficient in administering oils directly into the nasal passage tissues which could irritate the nasal tissues

There, thus exists the need for a more efficient dispenser of aromatic and other beneficial oils for personal use. It is an object of the present invention to provide a new and more hygienic system for dispensing aromatic and other beneficial oils to a person's nasal passages without touching the sensitive area of the nose.

### BRIEF DESCRIPTION OF THE DRAWINGS

The present invention will be understood and appreciated more fully from the following description taken in conjunction with the appended drawings in which:

FIG. 1 is a schematic illustration of a delivery system for dispensing oils, constructed and operative in accordance with an embodiment of the invention;

FIG. 2 is a sectional view of the scented inserts used with the delivery system of FIG. 1;

FIG. 3 is a perspective view of the nasal clip and scented inserts used with the delivery system of FIG. 1;

FIG. 4 is a side view of the delivery system of FIG. 1;

FIG. 5 is a bottom view of the delivery system of FIG. 1;

FIG. 6 Is a side view of nasal buds used with the delivery system of FIGS. 1-5; and

FIG. 7 is a perspective view of the scented inserts of FIG. 6.

### SUMMARY OF THE INVENTION

The present invention is directed to a system for delivering aromatic and other beneficial oils to the interior of a person's nose without the scented inserts containing oils touching the sensitive areas of the nasal passage.

There is thus provided, a delivery system, which comprises a nasal clip externally attached to a nasal septum of a user and a pair of scented inserts which are configured to fit and be secured by a pair of generally cylindrical shaped receptacles formed at the end of the nasal clip. The nasal clip and the pair of scented inserts are in close proximity to the septum of the user. The scented inserts are configured so as to avoid contact with the sensitive areas of the nasal passage.

Furthermore, in accordance with an embodiment of the invention, the nasal clip comprises a generally “U” shaped structure having a pair of generally cylindrical shaped receptacles formed at each free end of the “U” shaped structure.

Furthermore, in accordance with an embodiment of the invention each of said pair of generally cylindrical shaped receptacles is disposed to securely contain the scented insert.

Furthermore, in accordance with an embodiment of the invention, the scented inserts may be made from an absorbent polymer, such as ethylene vinyl acetate (EVA).

Furthermore, in accordance with an embodiment of the invention, the scented inserts are generally cylindrical having an indentation formed around the middle so that they snap into the cylindrically shaped receptacles.

Furthermore, in accordance with an embodiment of the invention, the cylindrically shaped receptacles are generally hollow, having a protrusion formed about its mid-section, said protrusion being configured to match the indentations of the scented inserts.

Furthermore, in accordance with an embodiment of the invention, the scented inserts are shorter in length than the cylindrically shaped receptacles.

Furthermore, in accordance with an embodiment of the invention, the scented inserts may be impregnated with any of a group of oils including aromatic oil, eucalyptus oil and cbd (cannabidiol) oil.

### DESCRIPTION OF THE PREFERRED EMBODIMENTS

The present invention relates to a delivery system for aromatic oils to a nasal clip.

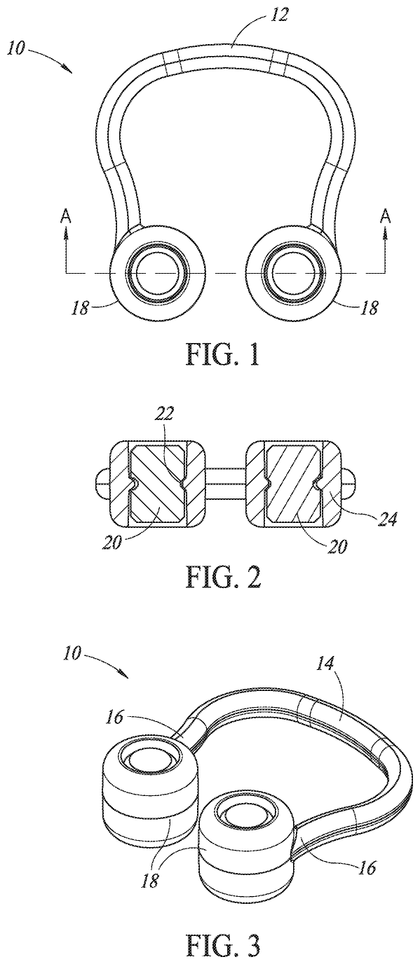

Reference is now made to FIGS. 1-7. FIG. 1 is a schematic illustration of the delivery system for aromatic oils, generally designated 10, constructed and operative in accordance an embodiment of the invention.

The delivery system comprises a nasal clip 12 for attachment to a nasal septum of a user and a pair of scented inserts 20 which are configured to fit the nasal clip 12.

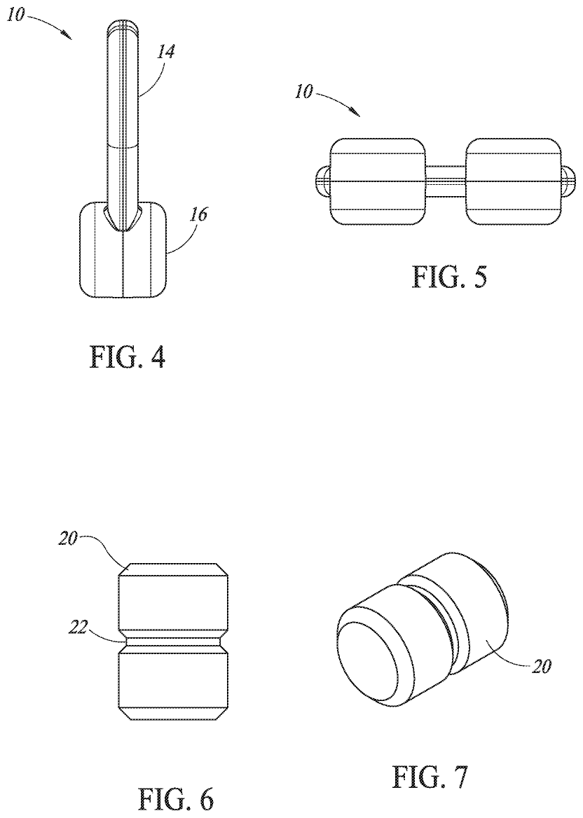

FIG. 2 is a sectional view of the scented inserts 20. FIG. 3 is a perspective view of the nasal clip 12 and scented inserts 20 used with the delivery system 10. FIGS. 4 and 5 are a side view and a bottom view, respectively of the delivery system 10. FIG. 6 Is a side view of scented inserts 20 and FIG. 7 is a perspective view of the scented inserts 20.

Nasal clip 12 comprises a generally “U” shaped structure 14 having a pair of free ends 16. A generally cylindrical shaped receptacle 18 is formed at each end of said free ends 16.

The nasal clip 12 and cylindrical shaped receptacle 18 may be formed by injection molding or any other method known in the art. The nasal clip 12 and cylindrical shaped receptacle 18 may be manufactured from food grade Polypropylene or similar.

The scented inserts 20 are cylindrically shaped to snuggly fit within each of the cylindrical shaped receptacles 18 and be secured by the receptacles 18.

The ends of the “U” shaped structure 14 of the nasal clip 12 are biased toward each other to grip the septum of the user. The cylindrical shaped receptacle 18 is inserted parallel to the septum.

The scented inserts 20 are preferably made of a polymer such as EVA (ethylene vinyl acetate), for example, which is highly absorbent. The scented inserts 20 are generally cylindrical having an indentation 22 (best seem in FIG. 6) formed around the middle so that they snap into shaped receptacles 18. The nasal clip 12 is externally attached to the septum of the user and, in contrast to prior art devices, cylindrical scented inserts are designed so that they do not come into contact with the sensitive mucous membranes of the nostrils.

The scented inserts 20 may contain an aromatic substance or other beneficial oil or substance. The scented inserts 20 are in close proximity to the septum of the user and thus, the scent (or aroma) is directed towards the user's nasal passages and inhaled during normal breathing activity.

The cylindrically shaped receptacles 18 are generally hollow, having a protrusion 24 formed about its mid-section (see FIG. 2), said protrusion 24 being configured to match the indentations 22 of the scented inserts 20, thereby to secure the scented inserts 20 within the shaped receptacles 18, so that they remain within the scented inserts 20 and are prevented from falling out. This unique snap-in feature makes the delivery system effective and safe.

The length of the scented inserts 20 may be configured to be shorter than the length of the cylindrical shaped receptacle 18. Being shorter adds an extra degree of safety to ensure that the scented inserts 20 do not touch the sensitive mucous membranes. Furthermore, the length of the scented inserts 20 are configured to be shorter so that leakage of the oils is prevented when inserted within the cylindrically shaped receptacles 18.

Though the embodiment above has been described with reference to aromatic oils, it will be appreciated by persons knowledgeable in the art, the delivery device may be used for the delivery of other types of oil, such as eucalyptus oils to ease nasal congestion and cbd (cannabidiol) oil found in the cannabis (or marijuana) plant which is known to reduce pain, anxiety and depression, for example.

It will be appreciated by persons knowledgeable in the art that the receptacle formed at each of the free ends of the nasal clip is not limited by the design shown in the drawing figures but may be varied in shape. For example, by adding a projection to the receptacle, as shown in the figures of the U.S. provisional application No. 62/724641, o incorporated herein by reference.

The scents may comprise essential oils of plants and fruits, many of which are known to be effective and beneficial to patients in many ways. Aromatherapy is helpful in improving a person's moods and emotions, for example.

Once exposed to air, the aromas in the polymer cylinders may last for 12 to 24 hours. A primary benefit of the delivery system is its portability. In contrast to aroma therapy where essential oils are diffused through a room through various methods, the delivery system allows a person to benefit from the aroma while on the go. Additional features of the nasal clip 12 include being discrete, hardly noticeable, ready to use out of the box, as well as its unique snap-in feature for safety. The scented cylinder inserts cannot be dislodged and cannot touch the sensitive mucous membranes in the nostrils.

Furthermore, the delivery system 10 containing a nasal clip 12 and scented inserts 20 is configured to be for one-time use and be disposable, which ensures that the delivery system 10 is hygienic.

It will be appreciated that the present invention is not limited by what has been described hereinabove and that numerous modifications, all of which fall within the scope of the present invention, exist. Rather the scope of the invention is defined by the claims, which follow:

1. A delivery system for dispensing oils comprising:
a nasal clip externally attached to a nasal septum of a user; and
a pair of scented inserts which are configured to fit and be secured by the nasal clip;
wherein said scented inserts are configured so that they do not come into contact with the sensitive mucous membranes of the nostrils.

2. The delivery system according to claim 1, wherein said nasal clip comprises a generally “U” shaped structure having a pair of generally cylindrical shaped receptacles formed at each free end of the “U” shaped structure.

3. The delivery system according to claim 2, wherein each of said pair of generally cylindrical shaped receptacles is disposed to securely contain the scented insert.

4. The delivery system according to claim 1, wherein said scented inserts are made from an absorbent polymer.

5. The delivery system according to claim 4, wherein said polymer comprises one of a group of polymers including ethylene vinyl acetate (EVA).

6. The delivery system according to claim 2, wherein said scented inserts are generally cylindrical having an indentation formed around the middle so that they snap into the cylindrically shaped receptacles.

7. The delivery system according to claim 6, wherein said cylindrically shaped receptacles are generally hollow, having a protrusion formed about its mid-section, said protrusion being configured to match the indentations of the scented inserts.

8. The delivery system according to claim 6, wherein said scented inserts are shorter in length than the cylindrically shaped receptacles.

9. The delivery system according to claim 1, wherein said scented inserts are impregnated with any of a group of oils including aromatic oil, eucalyptus oil and cbd (cannabidiol) oil.

10. The delivery system according to claim 1, wherein said nasal clip is a food grade Polypropylene.
